(19) United States
(12) Patent Application Publication
(10) Pub. No.: US 2008/0199902 A1
(43) Pub. Date: Aug. 21, 2008

(54) Biosensor For Determining The Biochemical Oxygen Demand (Bod) By Respirometry

(76) Inventors: Manuel Eduardo Young Anze, Valparaiso (CL); Cesar Raul Galindo Urra, Valparaiso (CL); Erika Mabel Valdes Carrion, Valparaiso (CL); Alejandro Mario Suarez Sotomayor, Valparaiso (CL); Fernando Antonio Albornoz Marquez, Valparaiso (CL); Cristian Andres Acevedo Gutierrez, Valparaiso (CL)

Correspondence Address: ERIC HANSCOM, 7395 PORTAGE WAY, CARLSBAD, CA, 92011, US

(21) Appl. No.: 11/814,000

(22) PCT Filed: Jan. 17, 2006
(86) PCT No.: PCT/EP2006/050235

§ 371 Date: Jan. 18, 2008

(30) Foreign Application Priority Data
Jan. 17, 2005 (CL) 84-2005

(51) Int. Cl. C12Q 1/02; C12M 1/00

(52) U.S. Cl. 435/29; 435/287.5

(57) ABSTRACT

A removable cartridge that contains an adjustable number of capsules inside of which there are adjustable masses of immobilized microorganisms, allowing the design of a simple, portable apparatus and the use of a reactor with less priming time. A circulating pump injects maintenance water into the reactor, and the used maintenance water is pumped out to a disposal location or device by the same pump. An air pump injects air into the maintenance liquid and the excess air is released through an airflow outlet. A dissolved oxygen sensor is used to take several measurements of the dissolved oxygen content of the sample water, and various formulas are used to analyze the results. Reactors of this type can be stored in cold allowing the replacement of the operating reactor easily.

### FIELD OF THE INVENTION

The present invention is related with the environmental characterization of liquid residue, as much as what refers to control and monitoring activities, as of treatment, more specifically with a biosensor for determining the biochemical oxygen demand (BOD) by respirometry.

### BACKGROUND OF THE INVENTION

The BOD is an important index for the organic pollution monitoring in liquids, therefore, is an indirect measure of the amount of organic material in liquids, in general and in water in particular, that can be biologically degraded by microorganism, since the dissolved oxygen get consumed during the degradation biochemical process of the organic material, then the said oxygen quantity can be expressed in equivalent form in terms of the quantity of required oxygen (respirometry), thus, the BOD is capable of identify in a quantitative way the degradable charge existing in residual water or in a body receptor.

Depending on temperature conditions, other nutrients availability and absence of inhibitors, the whole degradation process takes normally about 20 days. As a partial analytic measure, but statistically representative of the organic charge of a liquid residue, a conventional method denominated BOD₅ has been used that consist in incubating the sample for 5 days at 20° C., However this is a method that although is normalized, it is complicated and above all it duration of 5 days for quantifying the BOD does not allows to take opportune and efficient operational actions.

Consequently with the former, different types of biosensors have been developed based on respirometry and also associated methods that allows to know the BOD in situ and on real time , and obtain this measurement in a simple and fast form (in the order of minutes).

The biosensor consists on a combination of a transducer and a biological element, for example as microorganism and oxygen electrodes (see FIG. 1).

The on-line measurement systems consist basically in an unity (membrane or bioreactor), populated of microorganism (that can be specific of the liquid to be monitored) in which a continuous flow is maintained through a recirculating pump, being feed by peristaltical pumps that load the residual liquid simultaneously other pump saturates of oxygen the monitoring liquid (recipient fixed to the equipment or system), and a probe for the measurement of dissolved oxygen. This measurement can be made with different configurations.

By example immobilized microorganism on polyacrylamide gel and a oxygen electrode are used (“A rapid method for estimation of BOD by using Microbial Cells”, Isao Karube and cls., Biotechnology and Bioengineering Vol XIX , p.1535-1547, 1977).

Also the respirometric activity register of the microorganisms in a reactor is used, which are extracted in a little quantity (aliquot) from a chemostat.

STIP ISCO GmbH has a biosensor named biox 1010, for the measurement of the content of BOD and toxicity in an automatic form waters of different origin. The method used is respirometric, based on a microbial culture coming from the water to be monitored that gradually deposits over insoluble supports inside the reactor (EP 0369490), whose composition, concentration and activity are constant. It metabolizes the organic matter of the samples such that consumed oxygen for it oxidation allows to know the BOD and toxicity values.

The apparatus and method previously described have the difficult of containing a limited quantity of biomass that reduces the range of measurement to waters with low BOD. Is the case of those that use membranes supported over the oxygen sensor. On the other hand those based on microorganism supported over insoluble material, must be generated in situ over the base of the bacteria present in the liquid to be monitored making impossible design the microbiological load of the reactor and requiring priming time of many days that must be repeated every time that the biofilm get deteriorated because of use or toxicity.

In the case of apparatus based on feed from a chemostat, the operative requirements make necessary a great number of feeding pumps, empty, air, recirculation and wash generating highly complex equipment, sharing the limiting of priming time

### SUMMARY OF THE INVENTION

In the actual invention this problems are approached and for giving a solution it is proposed a removable cartridge that contains an adjustable number of capsules inside of which there are adjustable masses of immobilized microorganisms, allowing the design of a simple apparatus and the use of a reactor with less prime times. Reactors of this type can be stored in cold allowing the replacement of the operating reactor easily.

### BRIEF DESCRIPTION OF THE DRAWINGS

FIG. 1 (previous art), shows a general scheme of the constitutive parts of biosensors.

FIG. 2 shows the diagram of the system that measure BOD using the present invention.

FIG. 3 shows the Cartridge type removable bioreactor (BTC) of the present invention.

FIG. 4 shows a typical respirometric curve, indicating the distinct zones of respirometric activity.

FIG. 5 shows the results of a calibration BOD obtained in the laboratory versus the resulting respirometric area for each experiment using a standard solution.

FIG. 6 shows the results of a calibration BOD obtained in the laboratory versus the resulting respirometric area for each experiment using an effluent (RIL) solution coming from an industry.

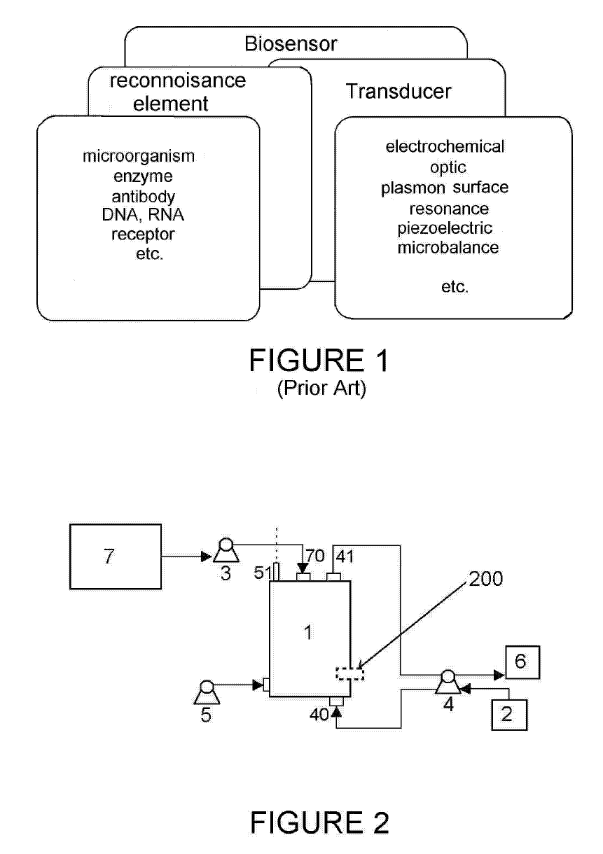

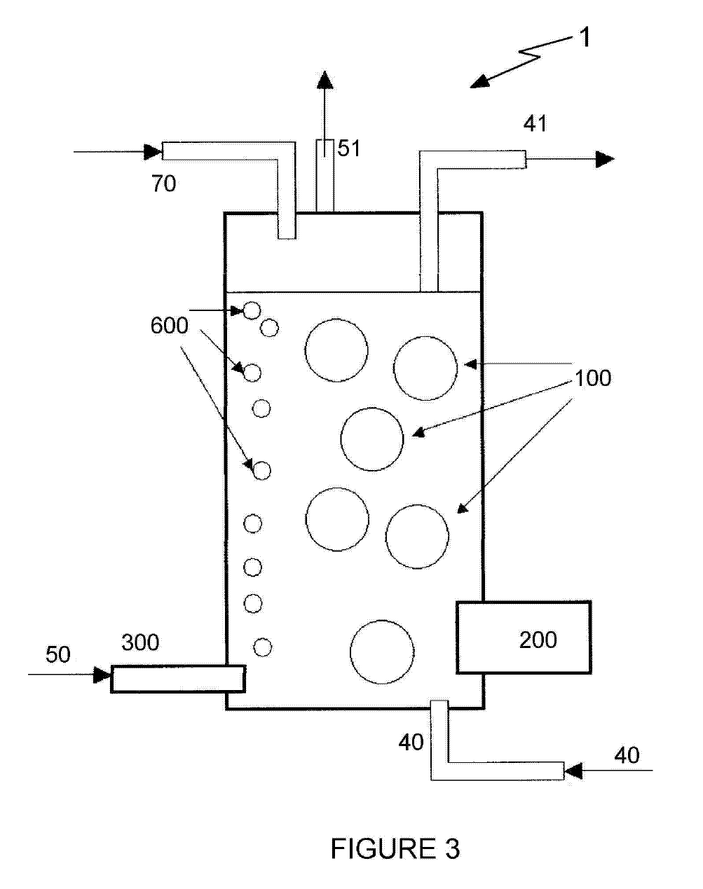

### DETAILED DESCRIPTION OF EMBODIMENTS OF THE INVENTION

In this way, the present invention provides a system that operates like a biosensor, for the fast measurement of DBO, where the apparatus used as respirometric element is a cartridge type removable and disposable bioreactor (BTC)(1), in which the biomass is encapsulated in a polymeric organic matrix suspended in a support solution which, in the preferred modality is calcium chloride.

As shown in FIG. 2, the BTC (1) has in every moment an inlet (40) and outlet (41) of support liquid, that comes from a recipient (2) that stores this support liquid. The circulating pump (4) maintains a constant flow, that is to say, the input and output flow are equal, while the liquid volume is constant inside of the BTC on every moment. The output support liquid is discarded, and can go to the drainage or other outlet (6), also has on every moment an air flow intake (50), that after bubbling discharges to the atmosphere (51).

In order to start the measurement, it is necessary to inject a sample, which is obtained from an aliquot of the canals, rivers or lakes (RILES) (7), inside the BTC (1) trough a second inlet (70). The injected sample is pulse type (the whole sample is injected at a time).

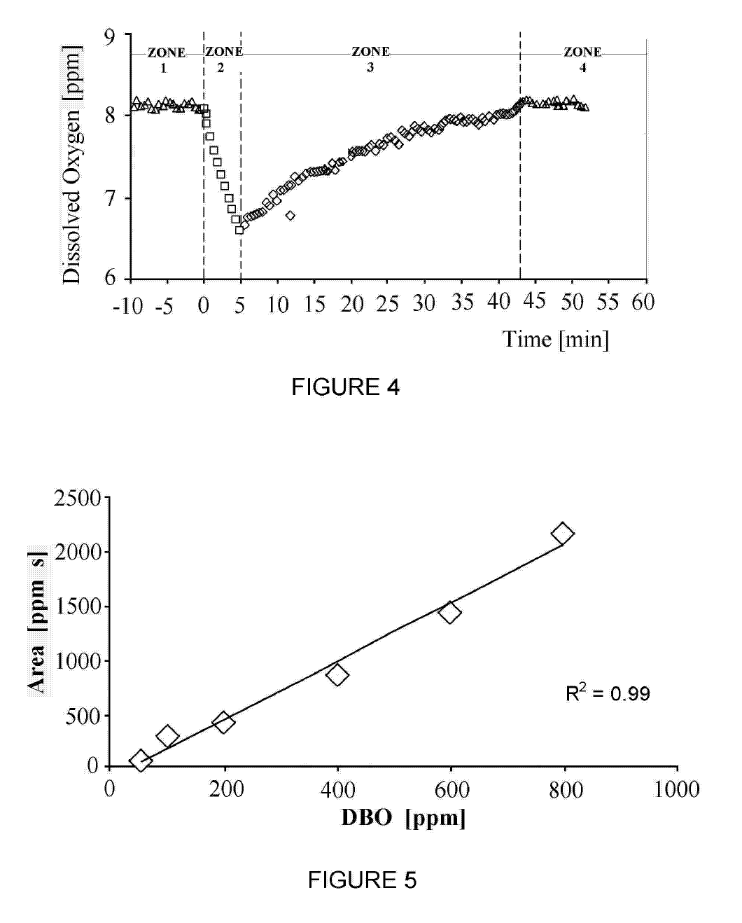

Before injecting the sample, the BTC is acquiring data from the dissolved oxygen sensor (200) to an acquiring data equipment, inside zone 1 (see FIG. 4), configuring a first base line. As shown on drawing 4, and when the sample is injected, the dissolved oxygen data begins to drop down, because of the injection of the sample coming from RIL (7), which marks the beginning of zone 2 denominate dissolved oxygen consumption zone. Then the dissolved oxygen begins to rise, which marks the beginning of zone 3 denominated dissolved oxygen recuperating zone. After a few minutes, the dissolved oxygen data get stabilized which indicates that a new base line is established, zone 4, in this way the BTC is ready for monitoring another sample to be injected.

Once the data is obtained, the dissolved Oxygen consumption and recovery zones data are selected, that is to say zones 2 and 3, where they are numerically integrated to obtain a first area under the curve (A₁).

Zones 1 and 4 are worked with the data of the respective base lines. An interpolation is done creating a theoretical base line corresponding to the dissolved oxygen consumption zone (zone 2) and the dissolved oxygen recovery zone (zone 3). The theoretical base line is integrated with which a second area under the curve is obtained (A₂).

To calculate the “Respirometric area” the resulting areas must be subtracted according to:

Respirometric area_{RIL}=A₂−A₁

The evaluation of BOD, can be done of different forms, one way is comparing the Respirometric Area_{RIL} versus the respirometric area of standard or known values of DBO measured previously.

$$BOD = \frac{Respirometric {area}_{RIL}}{Respirometric {area}_{STANDARD}} \cdot Factor$$

Other way is comparing the Respirometric area_{RIL} versus a calibrated curve previously constructed with standard known BOD solutions.

BOD=f(Respirometric area)

FIG. 5 shows the results of a BOD calibration obtained on laboratory versus the resulting respirometric area for each experiment using a liquid standard solution. The data is on table 1.

TABLE 1
BOD calibration obtained on laboratory

| | Area | BOD | BOD pattern |
|---|---|---|---|
| Sample | (ppm-O₂ s) | (ppm) | (ppm) |
| 1 | 57 | 57 | 50 |
| 2 | 282 | 141 | 100 |
| 3 | 426 | 194 | 200 |
| 4 | 865 | 358 | 400 |
| 5 | 1417 | 563 | 600 |
| 6 | 2152 | 836 | 800 |

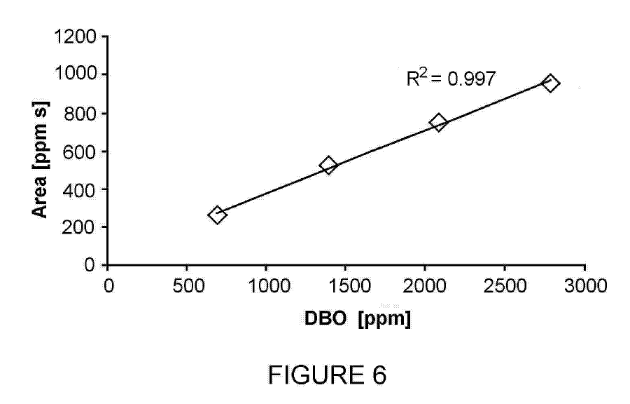

FIG. 6 illustrates the method for measuring BOD on real RILES samples coming from a beverage industry; data is shown on table 2.

TABLE 2
BOD measure on RILES samples of a beverage industry

| | Area | BOD | BOD Laboratory |
|---|---|---|---|
| Sample | (ppm-O₂ s) | (ppm) | (ppm) |
| 1 | 516 | 1544 | 1397 |
| 2 | 258 | 749 | 699 |
| 3 | 737 | 2224 | 2096 |
| 4 | 940 | 2850 | 2794 |

The cartridge type bioreactor (BTC) has an immobilized quantity of biomass on capsules (100), which was previously generated, that means, we have a preexistent biomass quantity that can be adjusted to the capsule (100), the capsule size (100) is also adjustable within certain limits, while the capsules number is also adjustable to the BTC size (1). All this indicates that the operation characteristics of the removable and disposable BTC (1) can be adjusted to the user requirements.

The BTC priming or activating time is less than the commercial alternatives available today, this prime or activating time goes from 4 hours to 1 day.

The BTC (1) is constructed from a cartridge of inert material with adapters for: inlet for feeding the support solution (40), outlet of liquid (41), measurement sample inlet (70), air inlet (50), air outlet (51), dissolved oxygen sensor (200) that in a preferred modality is removable. Additionally contains the capsules suspended on storing liquid or in support liquid when is in operation.

### EXAMPLE

The capsule forming solution consist on 1% sodium alginate where a population of bacteria Enterobacter sakasakii was introduced and previously homogenized and formerly isolated from residual water until reaching to a microbial concentration of 0.5 grams dry biomass/liter of capsule. This solution was dropped from the capsule device to a hardening solution of calcium chloride, 0.05 molar, generating capsules on the order of 1 millimeter diameter.

For constructing the BTC, 20 grams of capsules suspended on 40 cc of support liquid were used, consisting in a calcium chloride solution 0.05 molar.

The capsules were stored cold on trypticase soy (TSY) solution diluted on calcium chloride 0.05 molar solution.

1. A respirometry biosensor for fast determination of BOD (Biochemical Oxygen Demand) in water's flow, consisting of a removable cartridge type bioreactor (also known as a BTC), said cartridge type bioreactor comprising, a capsule-holding reservoir, a water sample inlet, a removable dissolved oxygen sensor (also known as a DOS), a DOS inlet, a liquid inlet, an air inlet, an air outlet, a water sample inlet, a liquid outlet, a supply reservoir, a circulating pump, where the water sample inlet is connected to a water sample pump which pumps water from a water sample in which a user of the invention desires to determine the BOD, where the air inlets allows an incoming quantity of air from an air pump to enter the BTC, where the incoming quantity of air forms a plurality of bubbles after entering the BTC through the air inlet and where an outgoing quantity of air exiting the BTC is discharged to the atmosphere through the air outlet, where the incoming quantity of air and the outgoing quantity of air are approximately equal in volume, where the circulating pump pumps a quantity of maintenance liquid from a supply reservoir through the liquid inlet into the BTC, where the liquid outlet serves as an opening in the BTC from which a quantity of used maintenance liquid can exit the BTC, where the circulating pump also pumps the quantity of used maintenance liquid from the BTC to a drainage reservoir, where the circulating pump maintains a constant flow, where the circulating pump keeps a level of liquid volume constant inside of the capsule holding reservoir of the BTC at all times; and where the BCT additionally comprises a quantity of biomass immobilized in one or more capsules, where the biomass comprises one or more microorganisms, where the one or more capsules are stored in the capsule holding reservoir, and where the BTC has a DOS inlet into which a DOS can be removably installed.

2. The respirometry biosensor according to claim 1, wherein the capsules are suspended in a solution.

3. The respirometry biosensor according to claim 2, wherein the solution is calcium chloride.

4. The respirometry biosensor according to claim 2, wherein the solution is a broth of trypticase soy (TSY) in calcium chloride solution.

5. The respirometry biosensor according to claim 2 wherein the one or more capsules are gels, where the gels comprise bacteria and a polymeric organic matrix.

6. The respirometry biosensor according to claim 5, wherein the polymeric organic matrix is an alginate-based solution.

7. The respirometry biosensor according to claim 6, wherein the one or more capsules can be adjusted in terms of one or more factors, the one or more factors selected from a group comprising number, size, and contents.

8. The respirometry biosensor according to claim 7, wherein the contents of the one or more capsules are adjustable to the characteristics of the water to be monitored.

9. The respirometry biosensor according to claim 8, wherein the characteristics of a water sample to be monitored can be used by a user of the invention to determine the quality and quantity of organic load.

10. The respirometry biosensor according to claim 9, wherein the water sample to be monitored comes from a source, where the source is selected from the group comprising: industrial plants, rivers, lakes and canals.

11. The respirometry biosensor according to claim 1, wherein the BCT is disposable.

12. The respirometry biosensor according to claim 1, wherein the quantity of biomass immobilized in the one or more capsules is previously generated biomass.

13. The respirometry biosensor according to claim 12, wherein the quantity of biomass quantity immobilized in the one or more capsules has been stored a refrigerated storage device which maintains the viability of the one or more microorganisms.

14. The respirometry biosensor according to claim 13, wherein the BTC is primed at least 4 hours.

15. The respirometry biosensor according to claim 14, wherein the BTC is primed for a time period that is no longer than 24 hours.

16. A respirometry biosensor for fast determination of BOD (Biochemical Oxygen Demand) in water's flow, consisting of a removable cartridge type bioreactor (also known as a BTC), said cartridge type bioreactor comprising, a capsule-holding reservoir, a water sample inlet, a removable dissolved oxygen sensor (also known as a DOS), a DOS inlet, a liquid inlet, an air inlet, an air outlet, a water sample inlet, a liquid outlet, a supply reservoir, a circulating pump, where the water sample inlet is connected to a water sample pump which pumps water from a water sample in which a user of the invention desires to determine the BOD, where the air inlets allows an incoming quantity of air from an air pump to enter the BTC, where the incoming quantity of air forms a plurality of bubbles after entering the BTC through the air inlet and where an outgoing quantity of air exiting the BTC is discharged to the atmosphere through the air outlet, where the incoming quantity of air and the outgoing quantity of air are approximately equal in volume, where the circulating pump pumps a quantity of maintenance liquid from a supply reservoir through the liquid inlet into the BTC, where the liquid outlet serves as an opening in the BTC from which a quantity of used maintenance liquid can exit the BTC, where the circulating pump also pumps the quantity of used maintenance liquid from the BTC to a drainage reservoir, where the circulating pump maintains a constant flow, where the circulating pump keeps a level of liquid volume constant inside of the capsule holding reservoir of the BTC at all times; and where the BCT additionally comprises a quantity of biomass immobilized in one or more capsules, where the biomass comprises one or more bacterias and an organic polymeric matrix, where the one or more capsules are stored in the capsule holding reservoir, and where the BTC has a DOS inlet into which a DOS can be removably installed.

17. The respirometry biosensor according to claim 16, wherein the capsules are suspended in a solution.

18. The respirometry biosensor according to claim 17, wherein the solution is calcium chloride.

19. The respirometry biosensor according to claim 17, wherein the solution is a broth of trypticase soy (TSY) in calcium chloride solution.

20. The respirometry biosensor according to claim 17 wherein the one or more capsules are gels, where the gels comprise bacteria and a polymeric organic matrix.

21. The respirometry biosensor according to claim 20, wherein the polymeric organic matrix is an alginate-based solution.

22. The respirometry biosensor according to claim 21, wherein the one or more capsules can be adjusted in terms of one or more factors, the one or more factors selected from a group comprising number, size, and contents.

23. The respirometry biosensor according to claim 22, wherein the contents of the one or more capsules are adjustable to the characteristics of the water to be monitored.

24. The respirometry biosensor according to claim 23, wherein the characteristics of a water sample to be monitored can be used by a user of the invention to determine the quality and quantity of organic load.

25. The respirometry biosensor according to claim 24, wherein the water sample to be monitored comes from a source, where the source is selected from the group comprising: industrial plants, rivers, lakes and canals.

26. The respirometry biosensor according to claim 16, wherein the BCT is disposable.

27. The respirometry biosensor according to claim 16, wherein the quantity of biomass immobilized in the one or more capsules is previously generated biomass.

28. The respirometry biosensor according to claim 27, wherein the quantity of biomass quantity immobilized in the one or more capsules has been stored a refrigerated storage device which maintains the viability of the one or more microorganisms.

29. The respirometry biosensor according to claim 28, wherein the BTC is primed at least 4 hours.

30. The respirometry biosensor according to claim 29, wherein the BTC is primed for a time period that is no longer than 24 hours.

31. A method for fast determining of BOD (Biochemical Oxygen Demand) in a flow of water, comprising the following steps:
a). obtain a respirometry biosensor for fast determination of BOD (Biochemical Oxygen Demand) in water's flow, where the respirometry biosensor consists of a removable cartridge type bioreactor (also known as a BTC), said cartridge type bioreactor comprising, a capsule-holding reservoir, a water sample inlet, a removable dissolved oxygen sensor (also known as a DOS), a DOS inlet, a liquid inlet, an air inlet, an air outlet, a water sample inlet, a liquid outlet, a supply reservoir, a circulating pump, where the water sample inlet is connected to a water sample pump which pumps water from a water sample in which a user of the invention desires to determine the BOD, where the air inlets allows an incoming quantity of air from an air pump to enter the BTC, where the incoming quantity of air forms a plurality of bubbles after entering the BTC through the air inlet and where an outgoing quantity of air exiting the BTC is discharged to the atmosphere through the air outlet, where the incoming quantity of air and the outgoing quantity of air are approximately equal in volume, where the circulating pump pumps a quantity of maintenance liquid from a supply reservoir through the liquid inlet into the BTC, where the liquid outlet serves as an opening in the BTC from which a quantity of used maintenance liquid can exit the BTC, where the circulating pump also pumps the quantity of used maintenance liquid from the BTC to a drainage reservoir, where the circulating pump maintains a constant flow, where the circulating pump keeps a level of liquid volume constant inside of the capsule holding reservoir of the BTC at all times; and where the BCT additionally comprises a quantity of biomass immobilized in one or more capsules, where the biomass comprises one or more bacterias and an organic polymeric matrix, where the one or more capsules are stored in the capsule holding reservoir, and where the BTC has a DOS inlet into which a DOS can be removably installed,
b). provide a removable dissolved oxygen sensor inside the BTC;
c) maintain a constant flow of maintenance liquid so the liquid volume is constant inside of the BTC at all times during its operation;
d). inject a quantity of air inside of the BTC;
e). acquire data from said removable DOS to an acquiring data device which is capable of configuring a first base line;
f).injecting a water sample to be monitored into the interior of the BTC;
g). register one or more values of dissolved oxygen from the DOS;
h). determine a second base line that is generated by the DOS when one or more values of dissolved oxygen get stabilized;
i). process the data obtained in step g); and
j). obtain one or more BOD values which can then be compared with known measurements of BOD.

32. The method according to claim 31, wherein the processing step obtained data of the step g) additionally comprises:
a) selecting one or more values of dissolved Oxygen consumption and recovery zones data wherein said data is numerically integrated to obtain a first area under a curve (A₁);
b) carry out an interpolation creating a theoretical base line corresponding to the dissolved oxygen consumption zone and the dissolved oxygen recovery zone;
c) Integrate the theoretical base line with which a second area is obtained under a curve (A₂); and
d) calculate a value for the “Respirometric area_{RIL}” by subtracting the area of A₁ from A₂, according to a formula: (A₂−A₁).

33. The method according to claim 32, wherein a step of obtaining BOD values and comparing them with known measurements of BOD is done according an equation:
$$BOD = \frac{Respirometric {area}_{RIL}}{Respirometric {area}_{STANDARD}} \cdot Factor$$

34. The method according to claim 32, wherein a step of obtaining BOD values compared with known measurements of BOD step is accomplished by a function:
BOD=f(Respirometric area)

35. The method according to claim 31, further comprising the step of previously generating the quantity of immobilized biomass in capsules.

36. The method according to claim 35, further comprising the step of cold storage of the quantity of biomass previously generated.

37. The method according to claim 31, further comprising the step of priming the BTC.

38. The method according to claim 37, wherein a priming time of said cartridge type reactor is at least 4 hours.

39. The method according to claim 38, wherein the priming time of said cartridge type reactor is no longer than 24 hours.

40. The method according to claim 31, wherein the step of injecting a water sample is done by injecting the entire sample at one time.

41. The method according to claim 31, wherein the organic polymeric matrix is an alginate based solution.

42. The method according to claim 31, wherein the one or more capsules can be adjusted in terms of one or more factors, the one or more factors selected from a group comprising number, size, and contents.

43. The method according to claim 42, wherein the contents of the one or more capsules are adjustable to the characteristics of the water to be monitored.
